(19) United States
(12) Patent Application Publication
(10) Pub. No.: US 2012/0029088 A1
(43) Pub. Date: Feb. 2, 2012

(54) ANTI-ANXIETY COMPOSITION

(75) Inventors: Hideaki HARA, Nara-shi (JP); Atsushi OOYAGI, Shizuoka-shi (JP); Takashi ISHIBASHI, Tokyo (JP)

(73) Assignee: JX NIPPON OIL & ENERGY CORPORATION, Tokyo (JP)

(21) Appl. No.: 13/188,328

(22) Filed: Jul. 21, 2011

(30) Foreign Application Priority Data
Jul. 27, 2010 (JP) 2010-168223

(51) Int. Cl. A61K 31/122; C07C 49/647; A61P 25/22; C07C 49/743

(52) U.S. Cl. 514/691; 568/367

(57) ABSTRACT

This invention provides a highly safe anti-anxiety composition that can be used for treatment or prevention of anxiety disorders. Such anti-anxiety composition comprises, as an active ingredient, a carotenoid.

### CROSS-REFERENCE TO RELATED APPLICATION

The present application claims priority of Japanese Patent Application No. 2010-168223 filed Jul. 27, 2010, the entire contents of which is incorporated herein by reference.

### TECHNICAL FIELD

The present invention relates to an anti-anxiety composition used for, for example, treatment or prevention of anxiety disorders.

### BACKGROUND ART

Anxiety disorders have heretofore been considered to be types of nervous diseases. Examples of representative symptoms of anxiety disorders include nervous disorders, mood disorders, personality disorders, behavior disorders, and sleep disorders. Approximately 50 products, such as benzodiazepines, thienodiazepines, and carbamate preparations, are known as pharmaceutical preparations used for treatment of the symptoms mentioned above.

Since long-term administration of such pharmaceutical preparations is required in order to improve symptoms, disadvantageously, use of such pharmaceutical preparations can cause severe side effects, such as drug dependence, motility disorders, and confusion, or minor side effects, such as drowsiness, dizziness, loss of appetite, and weakness. Accordingly, development of a novel anti-anxiety composition that can serve as an alternative to existing anti-anxiety agents has been awaited.

Astaxanthin, adonirubin, adonixanthin, canthaxanthin, and asteroidenone are members of the carotenoid family, and they are extensively present in animals, plants, and microorganisms. In particular, astaxanthin is known to have the effects such as antioxidation, anti-fatigue, anti-inflammatory, immunopotentiating, endurance-improving, and skin-beautifying effects (Astaxanthin no Kagaku (“Science of Astaxanthin”), written and edited by Kazunaga Yazawa, Seizando-Shoten Publishing Co., Ltd., November 2009).

Further, the possibility such that astaxanthin may be associated, as an active substance, with anti-stress action (JP Patent Publication (Kokai) No. H09-124470 A (1997)), memory improvement (JP Patent Publication (Kokai) No. 2001-2569 A), fatigue relief (JP Patent Publication (Kokai) No. 2006-16409 A), fatigue amelioration (JP Patent Publication (Kokai) No. 2006-347927 A), amelioration of cerebral dysfunctions (JP Patent Publication (Kokai) No. 2007-126455 A), and suppression of active oxygen generation in the brain (JP Patent Publication (Kokai) No. 2007-314436 A) is known.

However, it was not known in the past that carotenoids, such as astaxanthin, had anti-anxiety action.

### SUMMARY OF THE INVENTION

### Object to be Attained by the Invention

Under the above circumstances, it is an object of the present invention to provide a highly safe anti-anxiety composition.

### Means for Attaining the Object

The present inventors have conducted concentrated studies in order to attain the above object. As a result, the present inventors have found that carotenoid has anti-anxiety action, thereby completing the present invention.

The present invention includes the following.

(1) An anti-anxiety composition comprising, as an active ingredient, a carotenoid.

(2) The anti-anxiety composition according to (1), wherein the carotenoid is selected from the group consisting of astaxanthin, adonirubin, adonixanthin, canthaxanthin, and asteroidenone or a mixture of two or more thereof.

(3) The anti-anxiety composition according to (1) or (2), wherein the carotenoid is a mixture of astaxanthin and one or more carotenoids selected from the group consisting of adonirubin, adonixanthin, canthaxanthin, and asteroidenone.

(4) The anti-anxiety composition according to (1), wherein the carotenoid is astaxanthin.

(5) The anti-anxiety composition according to any one of (2) to (4), wherein the astaxanthin is a free form of astaxanthin.

(6) The anti-anxiety composition according to any one of (1) to (5), wherein the carotenoid is derived from Paracoccus carotinifaciens.

(7) A pharmaceutical preparation comprising the anti-anxiety composition according to any one of (1) to (6).

(6) A food or beverage product, functional food, or food additive comprising the anti-anxiety composition according to any one of (1) to (6).

(9) A feed comprising the anti-anxiety composition according to any one of (1) to (6).

### Effects of the Invention

The present invention can provide a highly safe anti-anxiety composition containing a carotenoid.

This description includes part or all of the contents as disclosed in the description and/or drawings of Japanese Patent Application No. 2010-168223, which is the priority document of the present application.

### BRIEF DESCRIPTION OF THE DRAWINGS

FIG. 1 shows a chart indicating the duration during which mice of the group to which a carotenoid mixture was administered remained within the open arms observed in the elevated plus-maze test of Example 1.

FIG. 2 shows a chart indicating the number of times mice of the group to which a carotenoid mixture was administered dipped their heads into holes (i.e., the number of head-dips) observed in the hole-board test of Example 2.

FIG. 3 shows a chart indicating the duration during which mice of the group to which a carotenoid mixture was administered dipped their heads into holes (i.e., the duration of head-dips) observed in the hole-board test of Example 2.

### EMBODIMENTS FOR CARRYING OUT THE INVENTION

Hereafter, the present invention is described in detail.

The anti-anxiety composition of the present invention comprises, as an active ingredient, a carotenoid. Anxiety disorders can be prevented or treated by having animals, such as humans, ingest the anti-anxiety composition of the present invention, or by administering the anti-anxiety composition thereto without side effects. As described above, the possibility that astaxanthin is associated, as an active substance, with anti-stress actions (JP Patent Publication (Kokai) No. H09-124470 A (1997)), memory improvement (JP Patent Publication (Kokai) No. 2001-2569 A), fatigue relief (JP Patent Publication (Kokai) No. 2006-16409 A), fatigue amelioration (JP Patent Publication (Kokai) No. 2006-347927 A), amelioration of cerebral dysfunctions (JP Patent Publication (Kokai) No. 2007-126455 A), and suppression of active oxygen generation in the brain (JP Patent Publication (Kokai) No. 2007-314436 A) is known. Based on the fact that experimental systems used for the evaluation of such action are different from those used for the evaluation of anti-anxiety action, the action that has heretofore been known is different from anti-anxiety action.

The term “anti-anxiety action (or anti-anxiety effect)” used herein refers to, for example, treatment, alleviation, or prevention of anxiety disorder conditions, such as nervous disorders, mood disorders, personality disorders, behavior disorders, and sleep disorders.

In the present invention, carotenoids are used as active ingredients. Examples of carotenoids include astaxanthin, adonirubin, adonixanthin, canthaxanthin, asteroidenone, and a carotenoid mixture of two or more thereof. Astaxanthin is particularly preferable. Alternatively, a mixture of astaxanthin and one or more carotenoids selected from among adonirubin, adonixanthin, canthaxanthin, and asteroidenone may be used as a carotenoid.

Further, examples of astaxanthin include free forms of astaxanthin and astaxanthin esters (e.g., monoesters and diesters), with the use of the free form of astaxanthin being preferable.

Commercially available carotenoids or carotenoids produced via conventional chemical synthesis techniques, microbial fermentation, extraction or purification from animals or plants, or other means (i.e., naturally occurring carotenoids) may be used.

Carotenoids produced from Paracoccus carotinifaciens via alcohol extraction with the use of ethanol or the like comprise, as a main ingredient, the free form of astaxanthin, as well as comprise adonirubin, adonixanthin, canthaxanthin, and asteroidenone, in addition to astaxanthin, and it can be used in the present invention. Alcohol extraction of carotenoid from carotenoid-producing microorganisms, including Paracoccus carotinifaciens, is carried out in accordance with the method described in, for example, JP Patent Publication (Kokai) No. 2009-50237 A. Specifically, the cultured microorganisms are suspended in a lower alcohol at 80° C. or higher (e.g., ethanol) or a combination of water and a lower alcohol at 80° C. or higher, carotenoid mixture is separated from microorganisms residue via filtration, a precipitate is obtained from carotenoid mixture via crystallization, and the precipitate is washed with a lower alcohol, followed by further washing with water according to need.

The anti-anxiety composition of the present invention can be prepared with the use of a carotenoid as an active ingredient as described above.

In addition to carotenoids, the anti-anxiety composition of the present invention can comprise pharmaceutically acceptable carriers (e.g., excipients and diluents) and additives that are adequately selected from among, for example, binders, fillers, lubricants, disintegrators, wetting agents, emulsifiers, buffers, suspending agents, preservatives, colorants, flavoring agents, and sweetening agents. Carriers and additives that are generally used for pharmaceutical preparations can be used for preparing the anti-anxiety composition of the present invention. Examples of binders include starch, polyvinyl pyrrolidone, and hydroxypropyl methylcellulose. Examples of fillers include lactose and microcrystalline cellulose. Examples of lubricants include talc, silica, and magnesium stearate. Examples of disintegrators include starch and sodium carboxymethyl starch. An example of a wetting agent is sodium lauryl sulfate. Examples of emulsifiers include cellulose derivatives and sorbitol. Examples of preservatives include methyl-p-hydroxybenzoate and sorbic acid. It should be noted that additives that can be used in the present invention are not limited to those mentioned above.

The anti-anxiety composition of the present invention can be prepared in the form of, for example, a pharmaceutical preparation for oral administration or parenteral administration (e.g., intravenous, intraarterial, intraperitoneal, transrectal, subcutaneous, intramuscular, sublingual, intranasal, or transvaginal administration). The forms of such pharmaceutical preparations are not particularly limited. Examples thereof include solutions, tablets, powders, granules, capsules, suppositories, sprays, controlled-release agents, suspensions, and drinks.

A dose of a carotenoid contained in the anti-anxiety composition of the present invention varies depending on factors, such as the age, body weight, sex, and conditions of a patient, in addition to severity of a patient. For example, a dose to be administered to an adult patient is 0.1 mg to 1 g and preferably 2 mg to 500 mg in terms of the free form of astaxanthin per day, although the dose is not limited to such range. According to need, such dose may be administered several separate times, such as 2 or 3 times. Also, the anti-anxiety composition of the present invention may be administered to a patient in combination with another anti-anxiety agent.

An effective amount of the anti-anxiety composition of the present invention may be added to or encapsulated into an arbitrary form, such as a tablet, capsule, granule, drink, or PET bottle. Alternatively, an effective amount of the anti-anxiety composition may be added to an arbitrary food or beverage product or functional food that does not substantially contain a carotenoid. Thus, the anti-anxiety composition of the present invention can be prepared in the form of a food or beverage product or functional food. Examples of food or beverage products and functional foods include, but are not limited to, confectioneries, retort pouch food, juice, teas, and dairy products. In addition, sweetening agents, seasonings, emulsifiers, suspending agents, antiseptics, or the like can be added to the food or beverage product or functional food, according to need. Further, the anti-anxiety composition of the present invention can be used as a food additive.

Also, an effective amount of the anti-anxiety composition of the present invention may be added to any forms of feed for livestock animals (e.g., horses, cattle, or pigs) or pet animals (e.g., cats or dogs) that do not substantially contain a carotenoid. Thus, the anti-anxiety composition of the present invention can be prepared in the form of feed. Anxiety disorders of animals can be prevented or treated via ingestion of such feed. Accordingly, such forms of feed are effective for animal breeding.

Pharmacological evaluation of the anti-anxiety composition of the present invention can be carried out using an elevated plus-maze test and a hole-board test involving the use of mice as described in the examples below. Such tests are generally employed for evaluation of anti-anxiety action, for example. In the case of the elevated plus-maze test, for example, the anti-anxiety composition of the present invention is evaluated as having satisfactory anti-anxiety action, when mice to which the anti-anxiety composition of the present invention has been administered remain within the open arms for a period of time significantly longer than that of mice to which the anti-anxiety composition has not been administered.

In the case of the hole-board test, the anti-anxiety composition of the present invention is evaluated as having satisfactory anti-anxiety action when mice to which the anti-anxiety composition of the present invention has been administered dip their heads into holes many times (i.e., number of head-dips) for long periods of time (i.e., duration of head-dips) at significant levels, compared with those of mice to which the anti-anxiety composition has not been administered.

### EXAMPLES

Hereafter, the present invention is described in greater detail with reference to the following examples, although the technical scope of the present invention is not limited to these examples.

### Preparation Example 1

### Preparation of Free Form of Astaxanthin

Astaxanthin was extracted from Paracoccus carotinifaciens with the use of ethanol. Ethanol extraction was carried out in accordance with JP Patent Application No. 2007-222476 (JP Patent Publication (Kokai) No. 2009-50237 A) or JP Patent Application No. 2009-046105 (JP Patent Publication (Kokai) No. 2010-193865 A), which was filed by the applicant of the present invention in the past. The obtained astaxanthin was in the free form, and content thereof was 67% by weight. Also, it contained carotenoids, such as adonirubin (12 wt. %), adonixanthin (6 wt %), canthaxanthin (1 wt %), and asteroidenone (less than 1 wt %), in addition to astaxanthin.

In the following examples, the product obtained above was used as a carotenoid mixture comprising astaxanthin as a main ingredient (i.e., the free form of astaxanthin).

### Example 1

### Evaluation of Anti-Anxiety Action of Free Form of Astaxanthin Via Elevated Plus-Maze Test

The anti-anxiety action of the free form of astaxanthin was evaluated using an apparatus for an elevated plus maze test in the following manner. As a comparative agent, a benzodiazepine anti-anxiety agent (diazepam) was used.

### 1-1. Experimentation Method

As experimental animals, 4-week-old ICR male mice (Japan SLC) were used. Mice were raised in separate cages under conditions in which they could freely ingest commercially available solid forms of feed and water. The conditions in the animal-raising chambers were designated as 12 hours in lightness and 12 hours in darkness (lightness: 8:00 a.m. to 8:00 p.m.), and temperature was set at 24° C.±2° C. All testing procedures were carried out from 10:00 a.m. to 6:00 p.m.

A carotenoid mixture comprising astaxanthin as a main ingredient was suspended in olive oil in an amount of 100 mg/kg-BW/day (per day per kg of mouse body weight) or 300 mg/kg-BW/day of the free form of astaxanthin, and the resulting suspension was administered orally to the test groups once a day for 10 days.

In contrast, olive oil was administered orally to the control group once a day for 10 days. Diazepam was dissolved in 0.5% CMC, and the resulting solution was administered once at a dose of 1.0 mg/kg-BW via intraperitoneal injection to a positive control group.

The groups to which astaxanthin had been administered and the control group were subjected to the elevated plus-maze test 1 hour after the final administration. Diazepam was administered via intraperitoneal injection to a positive control group 30 minutes before the initiation of the elevated plus-maze test. The elevated plus-maze test involved the use of 11 mice for the group to which 100 mg/kg-BW of astaxanthin had been administered, 21 mice for the group to which 300 mg/kg-BW of astaxanthin had been administered, 27 mice for the control group, and 7 mice for the positive control group.

The apparatus used for the elevated plus-maze test is composed of 2 open arms and 2 closed arms perpendicular to each other (30 cm (length)×5 cm (width) each) and a platform (5 cm×5 cm) at which the open arms intersect with the closed arms. The closed arms are provided with black side walls (15 cm (height)) and a gray floor, and the open arms are not provided with side walls but have a transparent floor. This apparatus was mounted at a height 50 cm from the floor.

Mice were placed on the central platform of the maze, and the duration during which the mice remained within the open arms was measured using an automatic apparatus for measuring movement (EthoVision XT; Noldus, Wageningen) for 10 minutes. The floor of the apparatus for the elevated plus-maze test is located at a high position, and the closed arms are surrounded by walls while the open arms are without boundaries. As the mice remain within the open arms for longer periods of time, accordingly, the sense of anxiety is alleviated.

The obtained record used the mean plus/minus the standard error to indicate results. Student's t test or Dunnett's test for multiple comparison was carried out, and the results were considered to be statistically significant when the p value was less than 0.05.

### 1-2. Results of Evaluation

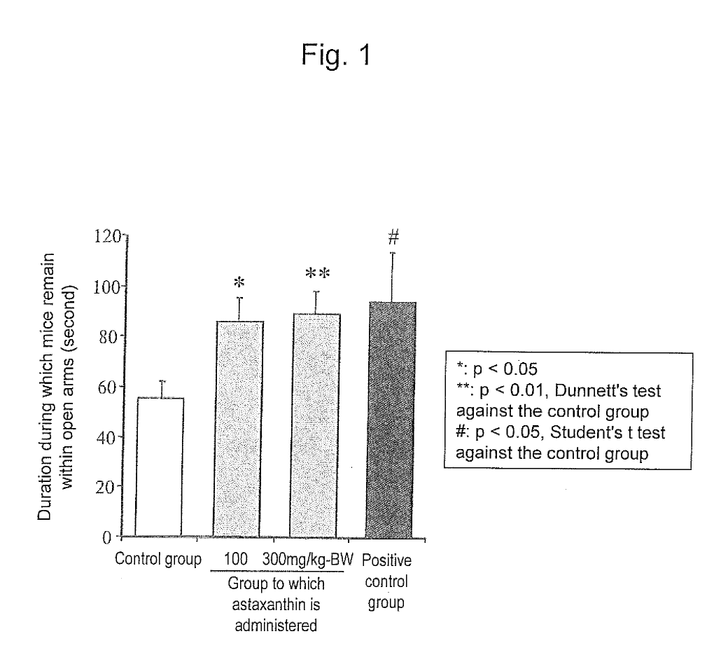

The results are shown in FIG. 1. FIG. 1 shows a chart indicating the duration during which mice of each group remained within the open arms.

When 100 mg/kg-BW of the free form of astaxanthin was administered, the duration during which mice remained within the open arms was prolonged to up to 1.6 times longer than the figure for the control group; this figure was also 92% of the figure for the positive control group, as shown in FIG. 1. When the free form of astaxanthin was administered in an amount of 300 mg/kg-BW, the duration was prolonged by approximately 2% compared with the case of administration of 100 mg/kg-BW, although no significant difference was observed.

As a result of multiple comparisons, a dose of 100 to 300 mg/kg-BW in terms of the free form of astaxanthin was found to produce significant anti-anxiety effects.

### Example 2

### Evaluation of Anti-Anxiety Action of Free Form of Astaxanthin Via Hole-Board Test

The anti-anxiety action of the free form of astaxanthin was evaluated using an apparatus for a hole-board test in the following manner. As a comparative agent, a benzodiazepine anti-anxiety agent (diazepam) was used.

### 2-1. Experimentation Method

As experimental animals, 4-week-old ICR male mice (Japan SLC) were used. Mice were raised in separate cages under conditions in which they could freely ingest commercially available solid feed and water. The conditions in the animal-raising chambers were designated as 12 hours in lightness and 12 hours in darkness (lightness: 8:00 a.m. to 8:00 p.m.), and temperature was set at 24° C.±2° C. All testing procedures were carried out from 10:00 a.m. to 6:00 p.m.

A carotenoid mixture comprising astaxanthin as a main ingredient was suspended in olive oil in an amount of 100 mg/kg-BW/day or 300 mg/kg-BW/day of the free form of astaxanthin, and the resulting suspension was administered orally to the test groups once a day for 10 days.

In contrast, olive oil was administered orally to the control group once a day for 10 days. Diazepam was dissolved in 0.5% CMC, and the resulting solution was administered once at a dose of 1.0 mg/kg-BW via intraperitoneal injection to a positive control group.

The groups to which astaxanthin had been administered and the control group were subjected to the hole-board test 1 hour after the final administration. Diazepam was administered via intraperitoneal injection to a positive control group 30 minutes before the initiation of the hole-board test. The hole-board test involved the use of 10 mice in the group to which 100 mg/kg-BW of astaxanthin had been administered, 10 mice in the group to which 300 mg/kg-BW of astaxanthin had been administered, 10 mice in the control group, and 4 mice in the positive control group.

The apparatus for the hole-board test is composed of a box without a top surface (30 cm (length)×30 cm (width)×16 cm (height)) provided with 4 holes each with a diameter of 2 cm on the floor surface.

Mice were placed at the center of the floor, and the number of times and the duration during which mice dipped their heads into holes were measured for 5 minutes. As the number of times of mice dipping their heads into holes increases and the duration becomes prolonged, the sense of anxiety is alleviated.

The obtained record used the mean plus/minus the standard error to indicate results. Student's t test or Dunnett's test for multiple comparison was carried out, and the results were considered to be statistically significant when the p value was less than 0.05.

### 2-2. Results of Evaluation

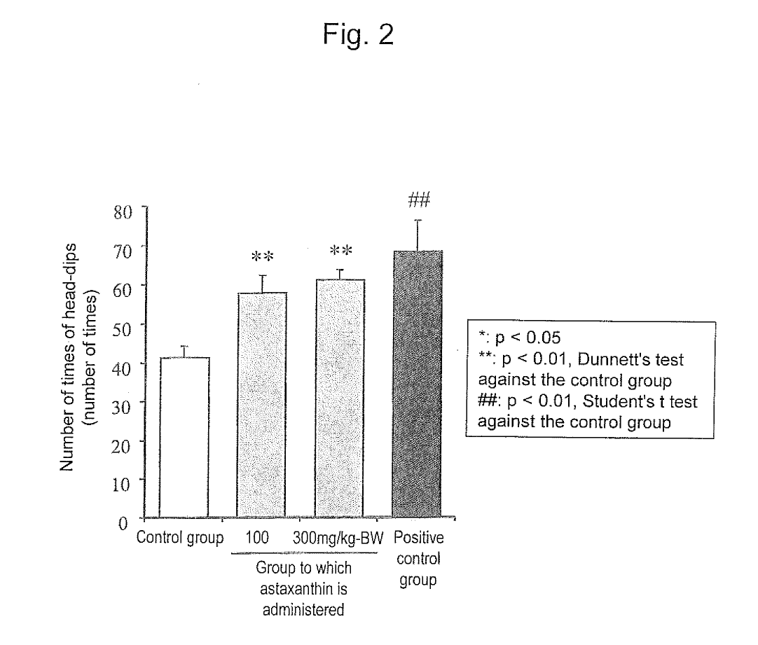

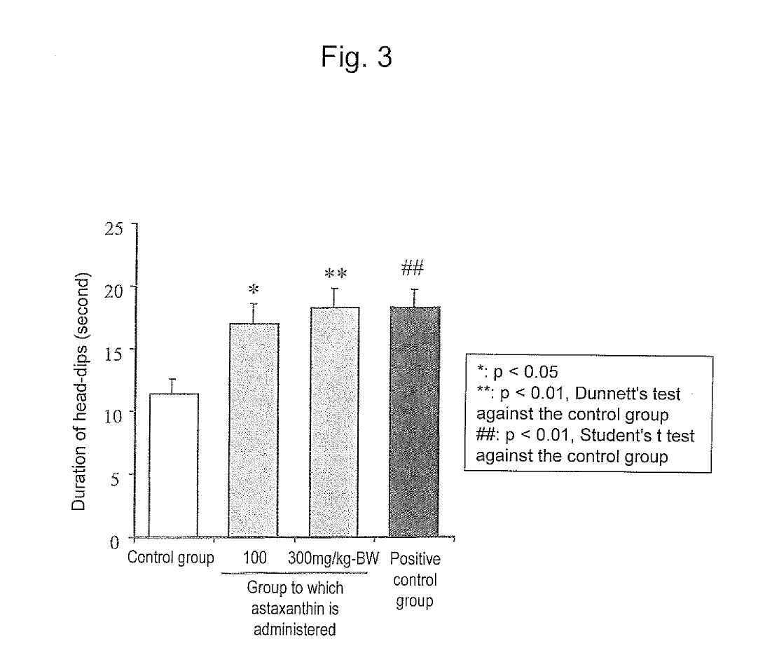

The results are shown in FIG. 2 and in FIG. 3. FIG. 2 shows a chart indicating the number of times mice of each group dipped their heads into holes (i.e., the number of head-dips). FIG. 3 shows a chart indicating the duration during which mice of each group dipped their heads into holes (i.e., the duration of head-dips).

When 100 mg/kg-BW of the free form of astaxanthin was administered, the number of head-dips was increased by 1.4 times or more than 1.4 times over the figure for the control group, and this figure was 87% of the figure for the positive control group, as shown in FIG. 2. When the free form of astaxanthin was administered in an amount of 300 mg/kg-BW, the number of times increased by approximately 4% compared with the case of administration of 100 mg/kg-BW, although no significant difference was observed. As a result of multiple comparisons, a dose of 100 to 300 mg/kg-BW in terms of the free form of astaxanthin was found to produce significant anti-anxiety effects.

When 100 mg/kg-BW of the free form of astaxanthin was administered, the duration of head-dips was prolonged to 1.5 times or longer than the figure for the control group, this figure was also 91% of the figure for the positive control group, as shown in FIG. 3. When the free form of astaxanthin was administered in an amount of 300 mg/kg-BW, the duration of head-dips was prolonged by approximately 4% compared with the case of administration of 100 mg/kg-BW, although no significant difference was observed. As a result of multiple comparisons, a dose of 100 to 300 mg/kg-BW in terms of the free form of astaxanthin was found to produce significant anti-anxiety effects.

All publications, patents, and patent applications cited herein are incorporated herein by reference in their entirety.

1. An anti-anxiety composition comprising, as an active ingredient, a carotenoid.

2. The anti-anxiety composition according to claim 1, wherein the carotenoid is selected from the group consisting of astaxanthin, adonirubin, adonixanthin, canthaxanthin, and asteroidenone or a mixture of two or more thereof.

3. The anti-anxiety composition according to claim 1 wherein the carotenoid is a mixture of astaxanthin and one or more carotenoids selected from the group consisting of adonirubin, adonixanthin, canthaxanthin, and asteroidenone.

4. The anti-anxiety composition according to claim 1, wherein the carotenoid is astaxanthin.

5. The anti-anxiety composition according to claim 2 wherein the astaxanthin is a free form of astaxanthin.

6. The anti-anxiety composition according to claim 1, wherein the carotenoid is derived from Paracoccus carotinifaciens.

7. A pharmaceutical preparation comprising the anti-anxiety composition according to claim 1.

8. A food or beverage product, functional food, or food additive comprising the anti-anxiety composition according to claim 1.

9. A feed comprising the anti-anxiety composition according to claim 1.

10. The anti-anxiety composition according to claim 2, wherein the carotenoid is a mixture of astaxanthin and one or more carotenoids selected from the group consisting of adonirubin, adonixanthin, canthaxanthin, and asteroidenone.

11. The anti-anxiety composition according to claim 3, wherein the astaxanthin is a free form of astaxanthin.

12. The anti-anxiety composition according to claim 4, wherein the astaxanthin is a free form of astaxanthin.

13. The anti-anxiety composition according to claim 2 wherein the carotenoid is derived from Paracoccus carotinifaciens.

14. The anti-anxiety composition according to claim 3, wherein the carotenoid is derived from Paracoccus carotinifaciens.

15. The anti-anxiety composition according to claim 4, wherein the carotenoid is derived from Paracoccus carotinifaciens.

16. The anti-anxiety composition according to claim 5, wherein the carotenoid is derived from Paracoccus carotinifaciens.

17. A pharmaceutical preparation comprising the an anxiety composition according to claim 2.

18. A pharmaceutical preparation comprising the anti-anxiety composition according to claim 3.

19. A food or beverage product, functional food, or food additive comprising the anti-anxiety composition according to claim 2.

20. A food or beverage product, functional food, or food additive comprising the anti-anxiety composition according to claim 3.
